(19) United States
(12) Patent Application Publication
(10) Pub. No.: US 2020/0157156 A1
(43) Pub. Date: May 21, 2020

(54) GRATELOUPIA CHIANGII-DERIVED LECTIN AND LECTIN GENE CODING FOR THE SAME

(71) Applicant: NATIONAL MARINE BIODIVERSITY INSTITUTE OF KOREA, Chungcheongnam-do (KR)

(72) Inventors: Jong Won HAN, Gunsan-si (KR); Hyun-Ju HWANG, Gunsan-si (KR); Hancheol JEON, Gunsan-si (KR); Jin-Woo HAN, Gunsan-si (KR)

(73) Assignee: NATIONAL MARINE BIODIVERSITY INSTITUTE OF KOREA, Chungcheongnam-do (KR)

(21) Appl. No.: 16/509,513

(22) Filed: Jul. 12, 2019

(30) Foreign Application Priority Data
Nov. 15, 2018 (KR) 10-2018-0140569

(51) Int. Cl. C07K 14/405; C07K 1/14; C07K 1/22

(52) U.S. Cl. CPC C07K 14/405; A61K 38/00; C07K 1/22; C07K 1/145

(57) ABSTRACT

A method of preparing an antiviral Grateloupia chiangii-derived lectin, includes preparing a crude extract of Grateloupia chiangii that is a red alga; and purifying a Grateloupia chiangii-derived lectin (GLC) from the crude extract by means of electrophoresis using D-mannose affinity chromatography. The present invention is expected to be useful in the field of medicine and pharmaceuticals in the future by identifying an amino acid sequence of the purified lectin and a complementary base sequence coding for the purified lectin.

### CROSS-REFERENCE TO RELATED APPLICATION

This application claims priority to and the benefit of Korean Patent Application No. 2018-0140569, filed Nov. 15, 2018, the disclosure of which is incorporated herein by reference in its entirety.

### BACKGROUND

### 1. Field of the Present Invention

The present invention relates to a certain protein derived from a natural product and the use thereof, and more particularly, to a lectin protein derived from a type of red alga, Grateloupia chiangii, and the use thereof.

### 2. Discussion of Related Art

Lectins are defined as carbohydrate-binding proteins, and were found approximately 100 years ago. Lectins have characteristics to recognize certain carbohydrate chains to agglutinate red blood cells. Therefore, this is referred to as agglutinin or hemagglutinin. The lectins are widely distributed in different organisms ranging from bacteria to plants and animals, and known to serve as molecules for recognition of vital activities. Owing to the carbohydrate-binding properties of lectins, the lectins have been considered to be probably applied in the biological research and pharmacology.

A red algal lectin has a unique primary structure, and thus has been proposed for various purposes as in the other lectins. Lectins present in the red algae are known to be mainly involved in the sexual reproduction. Although approximately 500 lectins have been screened from the red algae so far, only approximately 10 lectins have been purified from the red algae, and their amino acid sequences have been identified. This means that the red algal lectin was screened based on a hemagglutination test but the number of the red algal lectin for pharmacological applied research is very poor. For these reasons, applied research on lectins purified from the red algae has proceeded slowly due to the importance of the red algal lectin.

Meanwhile, Grateloupia chiangii is a red alga that usually grows in the southern coast and Jeju Island in Korea, but there is almost no research conducted on such a sugar-binding protein.

### SUMMARY OF THE PRESENT INVENTION

The present invention is technically designed to purify a novel lectin derived from a red alga and find its utility in consideration of the aforementioned circumstances, and therefore it is an object of the present invention to provide a method of isolating and purifying an antiviral lectin from Grateloupia chiangii that is a red alga.

It is another object of the present invention to provide a Grateloupia chiangii-derived lectin purified as described above and having antiviral properties.

It is still another object of the present invention to provide a lectin gene coding for the Grateloupia chiangii-derived lectin.

According to one exemplary embodiment of the present invention, the method of preparing an antiviral Grateloupia chiangii-derived lectin includes preparing a crude extract of Grateloupia chiangii that is a red alga; and purifying a Grateloupia chiangii-derived lectin (GCL) from the crude extract by means of D-mannose affinity chromatography.

The Grateloupia chiangii-derived lectin has antiviral properties so that it has a selective index (SI) of at least 28 and 140 against HSV1 and HSV2, respectively.

According to one exemplary embodiment of the present invention, the antiviral Grateloupia chiangii-derived lectin is a protein isolated and purified from Grateloupia chiangii, and has an amino acid sequence set forth in SEQ ID NO: 1.

According to one exemplary embodiment of the present invention, the lectin gene codes for the aforementioned antiviral Grateloupia chiangii-derived lectin, and has a base sequence set forth in SEQ ID NO: 2.

### BRIEF DESCRIPTION OF THE DRAWINGS

The above and other objects, features and advantages of the present invention will become more apparent to those of ordinary skill in the art by describing in detail exemplary embodiments thereof with reference to the attached drawings, in which:

FIG. 1 shows the electrophoresis results of a Grateloupia chiangii crude extract using D-mannose affinity chromatography;

FIG. 2 is a diagram showing the results of hemagglutination of GCL with respect to equine red blood cells and ovine red blood cells;

FIG. 3 is a diagram showing the results of hemagglutination assay on GCL with respect to carbohydrates and proteins;

FIG. 4 is a graph illustrating a degree of activity of GCL according to a temperature;

FIG. 5 is a graph for analyzing a change in degree of activity of CGL according to divalent cations added;

FIGS. 6 and 7 are graphs illustrating the results of glycan microarray assay on GCL;

FIG. 8 is a diagram showing an amino acid sequence of GCL and a complementary DNA sequence thereof;

FIGS. 9A-9H are graphs illustrating the LC-MS/MS data of GCL;

FIG. 10 is a diagram showing six repeat domain sequences of GCL; and

FIG. 11 is a conceptual diagram showing a tandem structure including the repeat domains of GCL.

### DETAILED DESCRIPTION OF EXEMPLARY EMBODIMENTS

Hereinafter, a method of preparing a Grateloupia chiangii-derived lectin according to the present invention and a lectin isolated and purified by the method will be described in detail. However, it should be understood that the following descriptions are provided to aid in understanding the present invention, but are not intended to limit the scope of the present invention. Accordingly, the scope of the present invention should be interpreted or defined solely by the appended claims.

Several red algal lectins have been reported to have unique structures and utilities, and research on a few marine red algae has been limitedly carried out. However, the present invention encompasses purifying a novel lectin from from a type of red alga, Grateloupia chiangii, and provides technical characteristics of the lectin. Hereinafter, the isolated lectin is referred to as a Grateloupia chiangii-derived lectin (GCL).

To purify GCL according to the present invention, first of all, a collected Grateloupia chiangii sample has to be washed with sterilized seawater and thoroughly dehydrated. The prepared sample is preferably stored at a temperature of at least −80° C. or lower for subsequent processes.

The prepared sample is immersed and pulverized in liquid nitrogen, and the like, added to a buffer solution in a powdered state, and then extracted therein. Then, the resulting extract is subjected to a subsequent purification process. Chromatography is performed by loading the crude extract on a D-mannose affinity resin. As will be described later, a single and clear fraction of GCL may be obtained by means of the chromatography.

Meanwhile, an amino acid sequence of the obtained GCL and a complementary DNA base sequence thereof may be identified by means of an analysis method and apparatus as will be described later. The amino acid sequence is set forth in SEQ ID NO: 1, and the DNA base sequence is set forth in SEQ ID NO: 2.

It is confirmed that the purified lectin (GCL) exhibits excellent antiviral properties, and thus the purified lectin (GCL), and it is thus estimated that the purified lectin (GCL) has very high medical and pharmaceutical utilities based on these characteristics.

Hereinafter, a method of isolating and purifying a Grateloupia chiangii-derived lectin according to one exemplary embodiment of the present invention, and the details of analysis of a purified lectin will be described. Meanwhile, the scope of the present invention is not restricted and limited to the following specific descriptions. In addition, the following descriptions may be made with reference to the accompanying drawings.

### EXAMPLES

Sample Collection

As a red alga, Grateloupia chiangii was collected in the southern coast of Korea. The collected sample was washed twice with sterilized seawater, and dehydrated with a paper towel. The washed sample was stored at −80° C. until use.

Lectin Purification

30 g (wet weight) of a sample was immersed in liquid nitrogen, and pulverized into fine powder using a mortar. A 5-fold volume of a buffer solution (Tris-buffered saline, 20 mM Tris-Cl, 150 mM NaCl, pH 7.5, TBS) was added to the sample, and stirred at 4° C. for 2 hours. The stirred solution was centrifuged at 4° C. for 20 minutes, and a supernatant was recovered as a crude extract.

The crude extract was directly loaded on a D-mannose affinity chromatography column using a Bio-rad FPLC system (Bio-rad, USA). The column was washed with a 10-fold volume of TBS to remove unbound proteins. GCL was eluted with a buffer solution including 0.5 M D-mannose, and its absorbance at 280 nm was monitored. Based on the electrophoresis (SDS-PAGE) results of the protein, a fraction having a single band was used for analysis.

The purified protein was dialyzed overnight with a TBS buffer solution while replacing a buffer solution every 4 hours. Total proteins and a concentration of a purified protein were determined by means of a Bradford protein microassay using an ELISA reader.

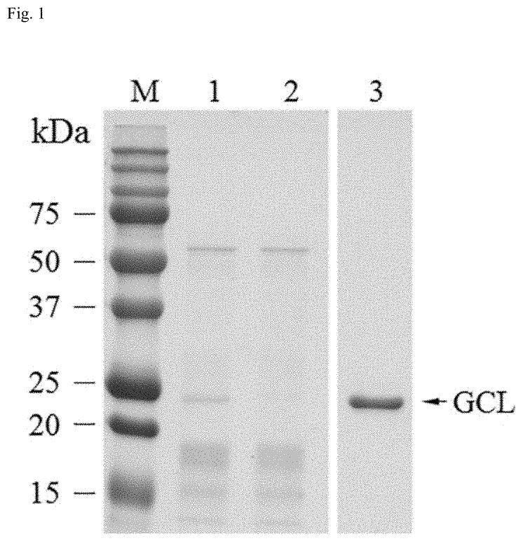

A mannose-bound protein, that is, a Grateloupia chiangii-derived lectin (GCL) was purified by means of mannose affinity chromatography, as described above. FIG. 1 shows the results of electrophoresis of the Grateloupia chiangii crude extract using D-mannose affinity chromatography (M: molecular weight marker; lane 1: crude extract; lane 2: flow-through fraction; and Lane 3: purified GCL). Referring to FIG. 1, GCL was observed in a fraction in which a single protein band was isolated without impurities. A molecular weight of GCL was approximately 25 kDa as observed on SDS-PAGE. As listed in Table 1 below, approximately 13.8 mg of the total proteins were extracted from 30 g of the Grateloupia chiangii sample, and 0.65 mg of GCL was obtained. The content of GCL amounted for 4.7% of the extracted total proteins, and had a degree of activity of 51,200 (titer/mg). The purification fold was increased to 14.72.

TABLE 1

| | Total | | Total | Specific | Percentage | |
|---|---|---|---|---|---|---|
| | protein | Concentration | activity | activity | of | Purification |
| | (mg) | (mg/mL) | (Titer) | (Titer/mg) | Recovery | fold |
| Crude extract | 13.8 | 0.092 | 48,000 | 3,478 | 100 | 1.00 |
| Affinity | 0.65 | 0.025 | 33,280 | 51,200 | 69.3 | 14.72 |
| chromatography | | | | | | |

Hemagglutination Assay and Carbohydrate Specificity

For a hemagglutination assay, horse and sheep blood was purchased (Hanil Comed, Korea). The blood was washed with phosphate buffered saline (PBS, pH 7.3) until a red color of a supernatant disappeared. Each of red blood cells was prepared into a 4% suspension in PBS. The sample was serially diluted in a 96-well U-bottom plate, and a 4% suspension of red blood cells was then added to each well. The sample was kept at room temperature for 30 minutes, and then confirmed for agglutination activities.

The carbohydrate specificity was determined by means of a hemagglutination inhibition test. Proteins and carbohydrates used for the inhibition test included D-glucose (D-Glc), D-mannose (D-Man), D-galactose (D-Gal), N-acetyl-D-glucosamine (GlcNAc), N-acetyl-D-galactosamine (GalNAc), D-fucose (F-Fuc), fructose (Fru), lactose (Lac), and fetuin. A sample (25 μL) having 4 haemagglutination activity units was mixed with each of the carbohydrates (25 μL), and 25 μL of a mixture was removed from each well. An equivalent amount of a 4% suspension of equine red blood cells was added to, and mixed with the sample. The plate was kept at room temperature for 30 minutes, and then confirmed for inhibitory activities.

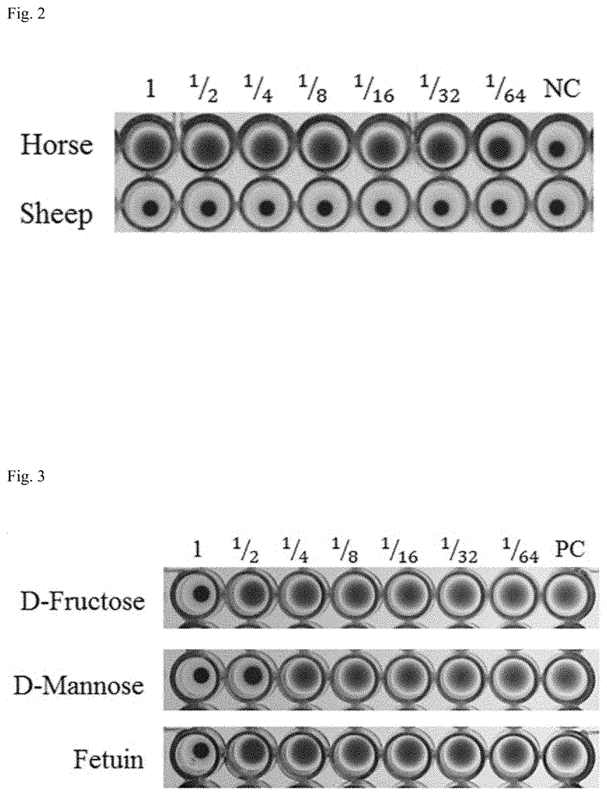

FIG. 2 is a diagram showing the results of hemagglutination of GCL with respect to equine red blood cells and ovine red blood cells. Referring to FIG. 2, the GCL agglutinated the equine red blood cells, but did not agglutinate the ovine red blood cells. A minimal concentration of the GCL for agglutination of the equine red blood cells was 0.8 μg/mL.

FIG. 3 is a diagram showing the results of hemagglutination assay on GCL with respect to carbohydrates and proteins. To determine the carbohydrate-binding specificity of lectin, an inhibition test by GLC was carried out using other 10 carbohydrates. Referring to FIG. 3, the haemagglutination activities of GCL 125 mM and 250 mM were inhibited by D-mannose and fructose, respectively. The activities were also inhibited by 195 μg/mL of a sugar protein (i.e., fetuin). However, the other carbohydrates tested did not inhibit the activities.

Effect of Temperature and Divalent Cation

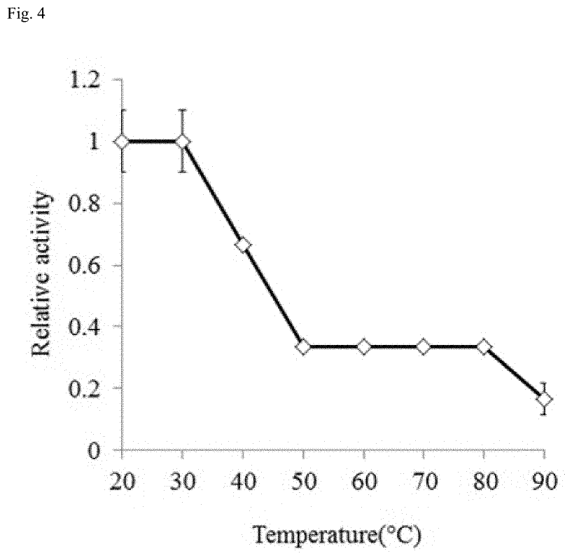

FIG. 4 is a graph illustrating a degree of activity of GCL according to a temperature. Referring to FIG. 4,

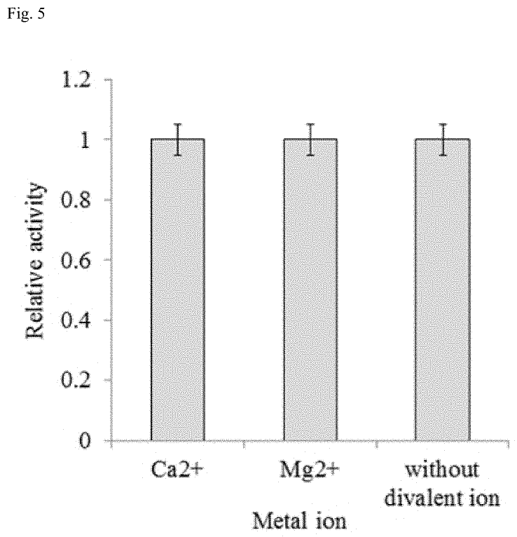

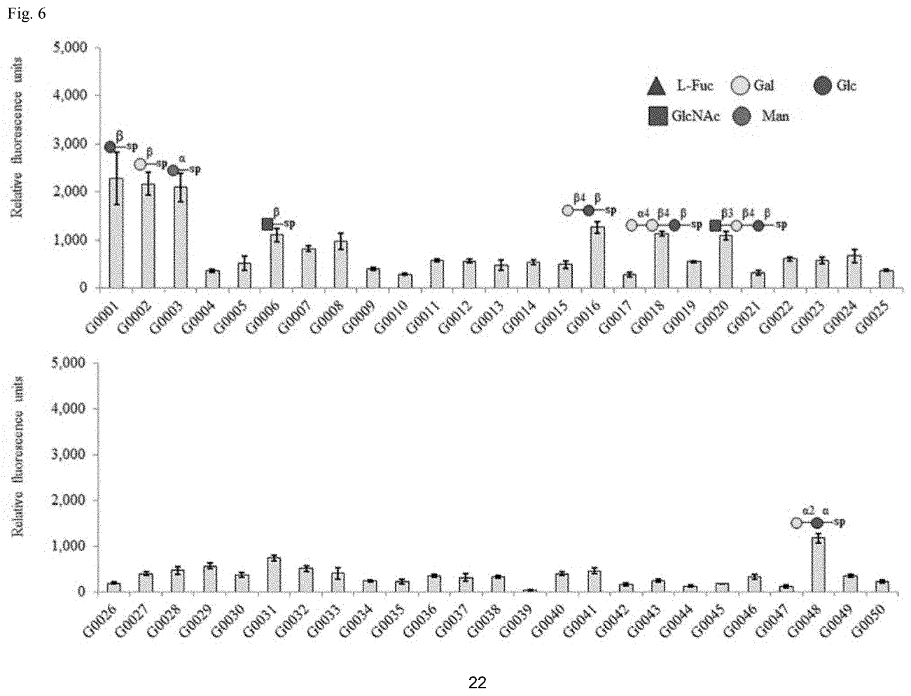

The thermal stability of GCL was measured at different temperatures (ranging from 20 to 90° C.). The GCL activities were not affected until the temperature reached 30° C. Half of the activities were lost at 40° C. Interestingly, approximately 15% of the initial activities were maintained even after the sample was heated at 90° C. for 30 minutes. FIG. 5 is a graph for analyzing a change in degree of activity of CGL according to divalent cations added. Referring to FIG. 5, the activities of lectin were not highly affected by the presence of the divalent cations. The addition of the divalent cations (Mg2⁺ and Ca2⁺) did not cause an increase in GCL activities.

Determination of N-Terminal Amino Acid Sequence of GCL

An N-terminal amino acid sequence was determined by the Korea Basic Science Institute (KBSI, Korea). A protein band was transferred to a PVDF membrane using a Western blot kit (Bio-rad, USA). The membrane was stained with a Ponceau S stain solution. A single band on the membrane was sliced into a piece using a knife, and then sent to the KBSI. A Procise 491 HT protein sequencer (Applied Biosystems, USA) was used to analyze the N-terminal sequence.

Peptide Mapping Using Mass Spectrometry

A protein band obtained through the electrophoresis was excised, digested with trypsin on the gel, and rinsed with Zip-tip (Millipore, Billerica, Mass., USA). The mass spectrometry was performed using 6545 Q-TOF LC/MS (Agilent Technologies, Santa Clara, Calif., USA) and Capillary LC-Nano ESI-MS. A ZORBAX 300SB-C8 column (1×50 mm, 3.5 μm; Agilent) was equilibrated with mass-grade water containing 0.1% (v/v) formic acid, and a sample was eluted with a gradient of water and 100% acetonitrile. A flow rate of a mobile phase was 10 μL/min. Tuning parameters used in the mass spectrometry were as follows.

[Capillary temperature: 300° C.; Source voltage: 1.9 kV; Skimmer voltage: 45 V; and Fragment voltage: 175 V]

Cloning of GCL and Determination of cDNA Sequence

Based on the N-terminal sequence and peptide mapping results, a cDNA sequence was obtained in comparison with the transcriptome data (generated by Hi-seq 3000). The full-length cDNA sequence was determined by PCR. Total RNAs were obtained using a Qiagen Plant total RNA isolation kit according to the manufacturer's method. The quality of the total RNAs was determined using a spectrophotometer and formaldehyde agarose gel electrophoresis. A cDNA synthesis kit was used to synthesize the first strand cDNA. The cDNA was purified using an Intron PCR purification kit, and the purified cDNA was directly used for PCR. PCR primers were designed based on the transcriptome data and the N-terminal sequence results. A PCR reaction was performed as follows.

Predenaturation was performed at 95° C. for 2 minutes, 35 cycles of 95° C. for 20 seconds, 60° C. for 30 seconds, and 72° C. for 1 minute, followed by a final reaction at 72° C. for 10 minutes. PCR products were confirmed through the agarose gel electrophoresis, a target band was excised with a sharp knife, and used for purification. The PCR product was purified using a gel elution kit according to the manufacturer's method. The isolated PCR product was cloned into a T-easy cloning vector, and host DH5α was transformed with the cloning vector. The transformants were plated on a LB-agar plate containing 100 μg/mL of ampicillin, and incubated overnight at 37° C. After the incubation, positive colonies were collected, and incubated overnight at 37° C. in a LB broth. A plasmid was isolated using a Qiagen plasmid isolation kit (Qiagen, USA). DNA was sequenced using a Sanger-based method (Macrogen, Korea).

Glycan Microarray

A glycan microarray was performed by Ebiogen (Korea). A glycan array kit was purchased from RayBioTech (Norcross, Ga., USA). An array consisting of 300 synthetic glycans printed in quadruplicate on a glass slide was used. Label-based detection was performed according to the manufacturer's protocol. 50 μg/mL of biotinylated lectin was put into wells, and gently shaken for 3 hours or more. The glass slide was washed with 1× washing buffers I and II provided in the kit. The glycan-lectin binding was detected by reacting with Cy3 equivalent dye-conjugated streptavidin at room temperature for an hour. Signals for Cy3 detection were visualized using a microarray laser scanner (Genfix 4100A, Molecular Devices, Sunnyvale, Calif., USA) (excitation at 554 nm, and emission at 568 nm). Data extraction was performed using microarray assay software Genfix. The R,FF;ZLS array data was analyzed using RayBioTech (RayBioTech) software.

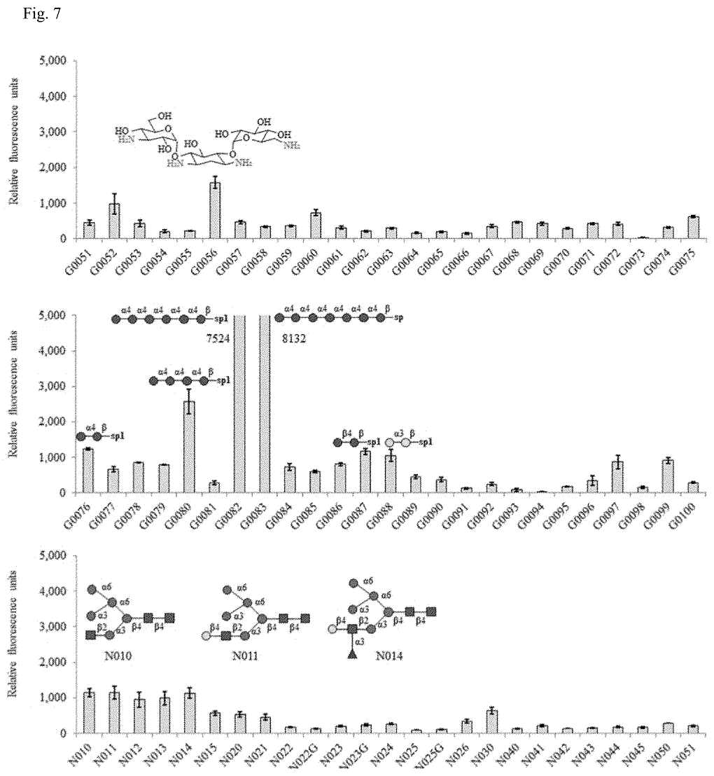

FIGS. 6 and 7 are graphs illustrating the results of glycan microarray assay on GCL.

To determine the carbohydrate specificity, a glycan microarray was carried out using a glycan-300 array kit in which 300 different carbohydrates were immobilized. Referring to FIGS. 6 and 7 and Table 2 below, 18 of the 300 tested carbohydrates produced at least 1,000 positive signals. GCL produced weak binding signals to the monosaccharides, that is, β-Glc-sp, β-Gal-sp, and α-Man-sp. Normalized signal intensities were 2,579, 2,269, and 2,194, respectively. Also, the GCL was able to bind to maltotetraose-β-Sp1 with a signal intensity of 2,705. The GCL was strongly bound to maltohexaose-β-Sp1 and maltoheptaose-β-Sp1. This protein was able to bind to N-glycan having a weak biding affinity (a signal intensity of 1,000). It was that high-mannan structures, that is, Man-α-1,6-(Man-α-1,3-), Man-α-1,6-(GlcNAc-β-1,2-Man-α-1,3-)Man-β-1,4-GlcNAc-β-1, and 4-GlcNAc-Sp5, interacted with the GCL.

TABLE 2

| | | |
|---|---|---|
| | | RFU |
| | | (Normalized) |
| No. | Glycan structure | GCL |

Monosaccharides

| | | |
|---|---|---|
| G0001 | β-Glc-Sp | 2,273 ± 544 |
| G0002 | β-Gal-Sp | 2,165 ± 237 |
| G0003 | α-Man-Sp | 2,093 ± 294 |
| G0006 | β-GlcNAc-Sp | 1,104 ± 133 |

Disaccharides

| | | |
|---|---|---|
| G0016 | Gal-β-1,4-Glc-β-Sp | 1,260 ± 126 |
| G0048 | Glc-α-1,2-Gal-α-Sp | 1,173 ± 110 |
| G0076 | Glc-α-1,4-Glc-β-Sp1 | 1,238 ± 28 |
| G0087 | D-cellose-β-Sp1 | 1,165 ± 82 |

Globo series, Milk Oligosaccharides and GAGs

| | | |
|---|---|---|
| G0018 | Gal-α-1,4-Gal-β-1,4-Glc-β-Sp | 1,124 ± 47 |
| G0020 | GalNAc-β-1,3-Gal-β-1,4-Glc-β-Sp | l,092 ± 92 |

Amino Glycoside

| | | |
|---|---|---|
| G0056 | Sisomicin Sulfate | 1,583 ± 177 |

Natural Oligosaccharides

| | | |
|---|---|---|
| G0080 | Maltotetraose-β-Sp1 | 2,577 ± 351 |
| G0082 | Maltohexaose-β-Sp1 | 7,524 ± 1,976 |
| G0083 | Maltoheptaose-β-Sp1 | 8,132 ± 1,810 |

N-glycans

| | | |
|---|---|---|
| N-010 | Man-α-1,6-(Man-α-1,3-)Man-α-1,6- | 1142 ± 116 |
| | (GlcNAc-β-1,2-Man-α-1,3-)Man-β- | |
| | 1,4-GlcNAc-β-1,4-GlcNAc-Sp5 | |
| N-011 | Man-α-1,6-(Man-α-1,3-)Man-α-1,6- | 1148 ± 173 |
| | (Gal-β-1,4-GlcNAc-β-1,2-Man-α-1,3-)Man- | |
| | β-1,4-GlcNAc-β-1,4-GlcNAc-Sp5 | |
| N-014 | Man-α-1,6-(Man-α-1,3-)Man-α-1,6- | 1129 ± 151 |
| | [Gal-β-1,4-(Fuc-α-1,3-)GlcNAc-β- | |
| | 1,2-Man-α-1,3-]Man-β-1,4-GlcNAc- | |
| | β-1,4-GlcNAc-Sp5 | |

Human Milk Oligosaccharides

| | | |
|---|---|---|
| H0400 | Gal-β-1,4-Glc-Sp | 1,044 ± 110 |

Molecular Characterization and Cloning of dGCL

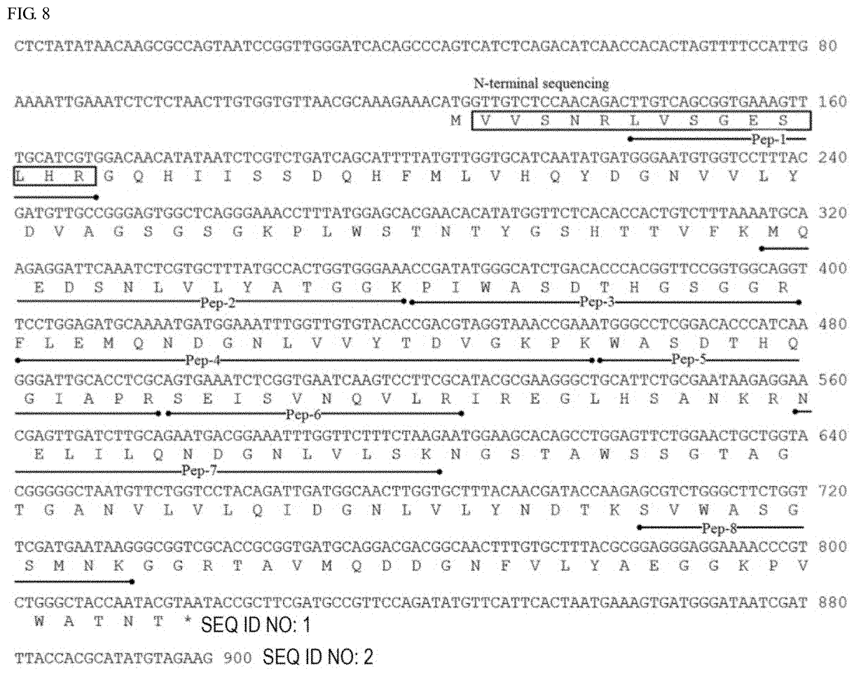

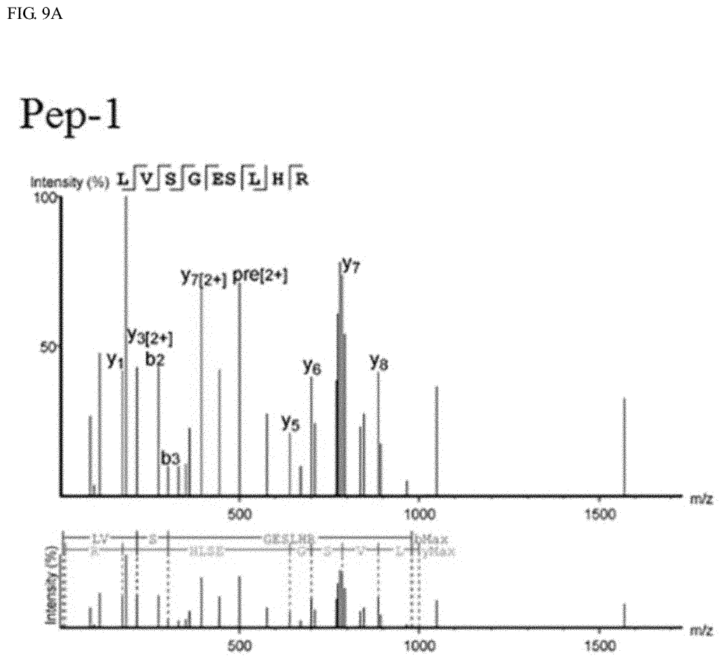

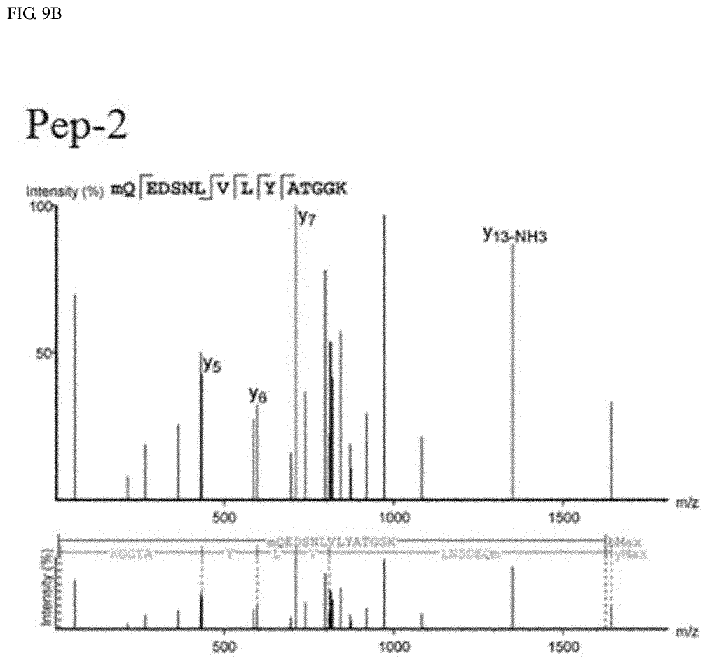

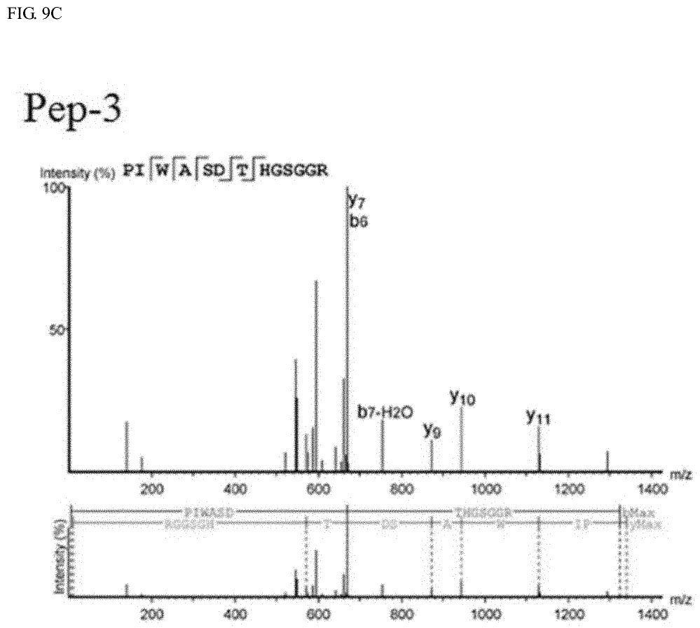

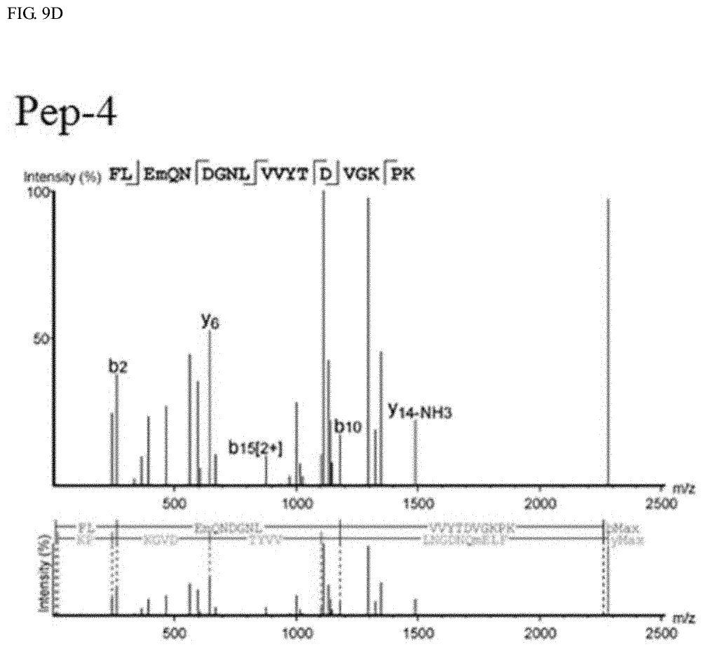

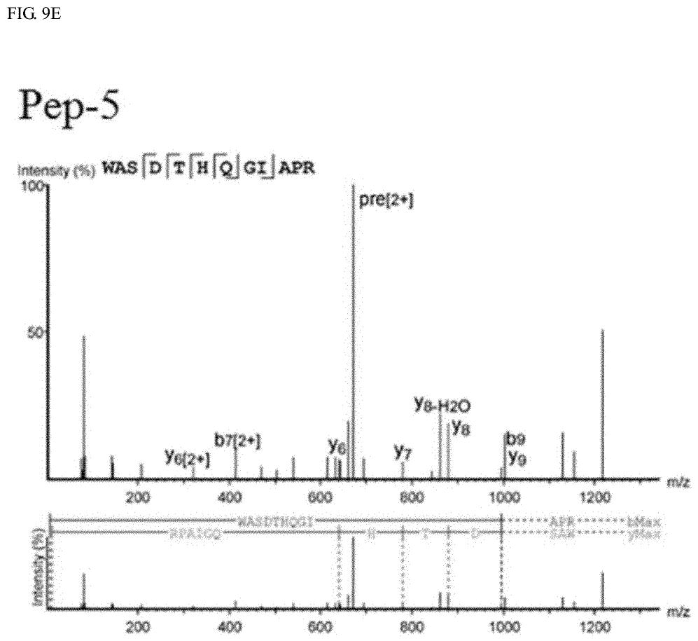

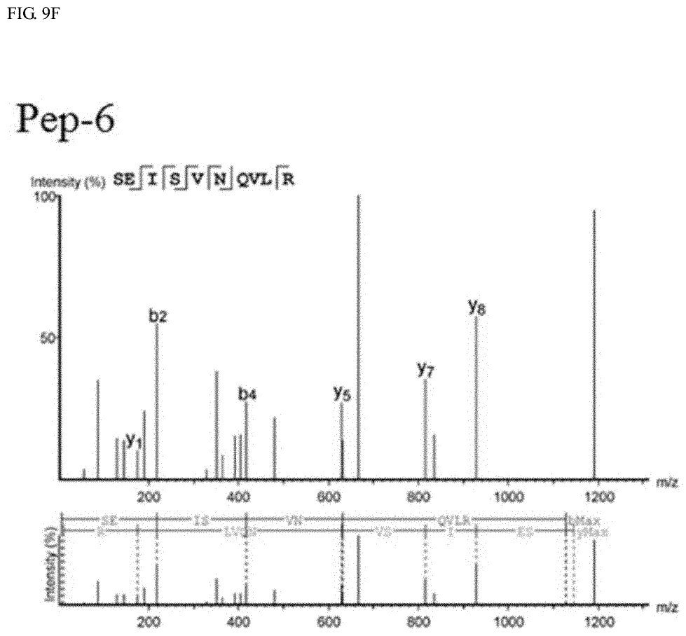

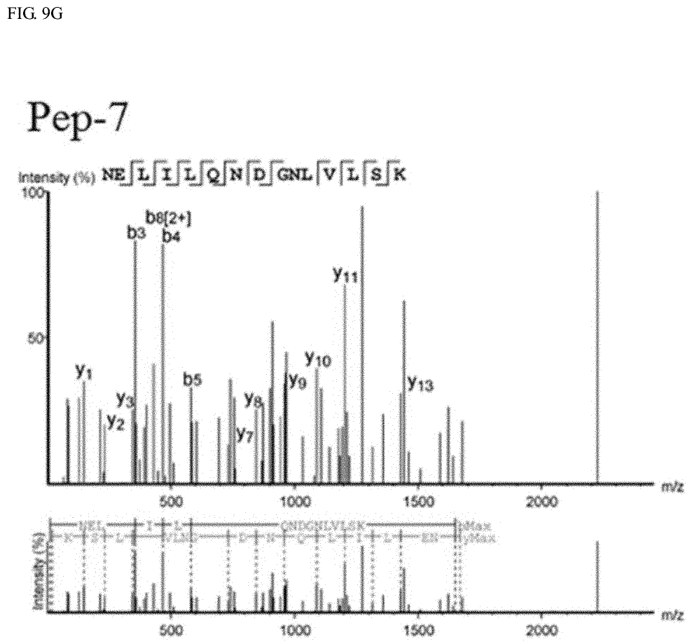

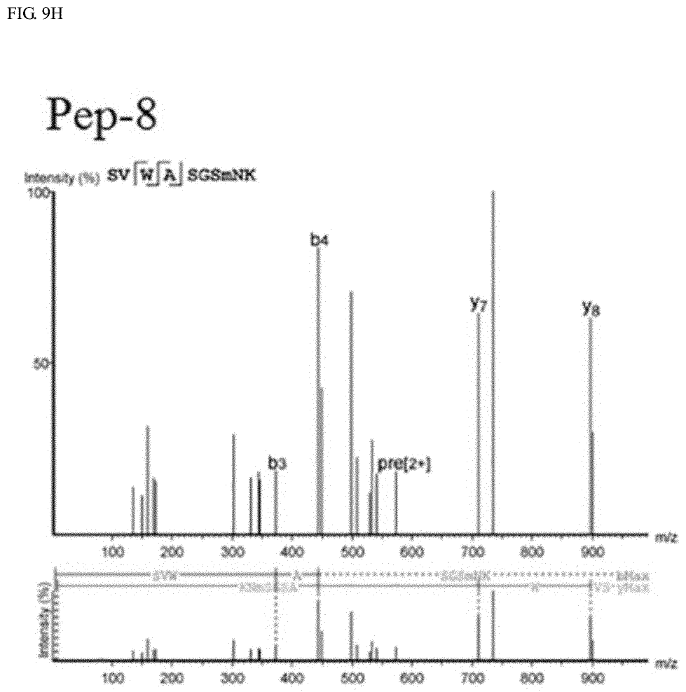

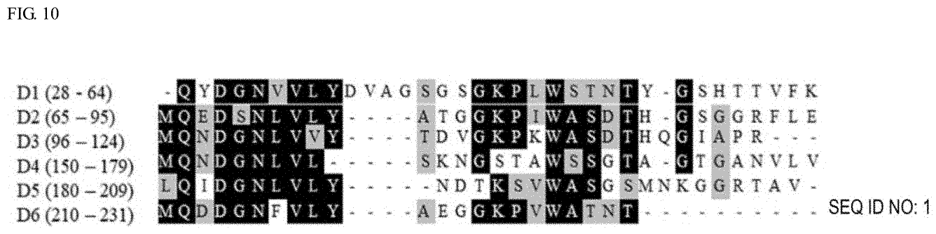

FIG. 8 is a diagram showing an amino acid sequence of GCL and a complementary DNA sequence thereof. FIGS. 9A-9H are graphs for analyzing eight peptides constituting a GCL protein. An N-terminal amino acid sequence of GCL was determined using an Edman degradation method. Referring to FIG. 8, it was confirmed that the N-terminal sequence was Val-Val-Ser-Asn-Arg-Lue-Val-Ser-Gly-Glu-X-Leu-His-Arg. The full-length cDNA sequence was determined by PCR and transcriptome data generated using Hi-seq 2000. cDNA consisted of 900 bp, and contained a 696 bp open reading frame (ORF). The protein had a calculated molecular weight of 24.9 kDa and a theoretical isoelectric point (pI) of 6.97. FIGS. 9A-9H are graphs illustrating the LC-MS/MS data of GCL. The mass data of the eight peptides was obtained from the LC-MS/MS data, and the mass of the peptides generally amounted for approximately 70% of the protein sequence. The aforementioned peptide mapping data and N-terminal sequence analysis results were the expected peptide-mass information. N-terminal methionine was judged to be removed after a post-transcriptional modification through the data comparison with the N-terminal sequence. FIG. 10 is a diagram showing six repeat domain sequences of GCL. FIG. 11 is a conceptual diagram showing a tandem structure including the repeat domains of GCL. Referring to FIGS. 10 and 11, it was interestingly confirmed that the GCL had a tandem repeat structure including the six repeat domains consisting of approximately 30-mer amino acids. The GCL was a member of this lectin B class, and had similarity to mannose-binding lectins derived from the bacteria. A dimerization interface with a mannose-binding domain was highly conserved. However, similar proteins from a group of plants or algae were not found using the GenBank database.

Antiviral Effect

Referring to Table 3, the GCL had an antiviral effect against herpes simplex virus 1 (HSV1) and herpes simplex virus 2 (HSV2) when present at low concentrations of 0.18 μg/mL and 0.036 μg/mL, respectively. Also, the GCL did not exhibit cytotoxicity when it reached 5 μg/mL. Concanavalin A, which was a representative mannose-binding protein used as the control, exhibited cytotoxicity at 29.29 μg/mL, and had an antiviral effect at 5 μg/mL or less. The antiviral effect was determined through cell viability, and the like using a virus-induced cytopathic effect (CPE) inhibition method and an MTT method. As antiviral drugs used for infection and treatment of herpes simplex virus, Acyclovir, dextran sulfate 8000, and pentosan polysulfate were used as the positive controls. Among the positive controls used, Acyclovir exhibited the highest antiviral activities, and had degrees of activity of 0.52 μM and 2.87 μM against HSV1 and HSV2, respectively. The GCL had an antiviral activity at a concentration lower than the positive controls used. Considering that the further antiviral research proceeded when a selective index (SI) value is greater than or equal to 5, which indicates a high potential for development as the drug, the GCL was also judged to have the probability as potential antiviral preparations because it had selective indexes of 28 and 140 or more, respectively.

TABLE 3

| | | Antiviral activity | | Selective | |
|---|---|---|---|---|---|
| | | (EC₅₀; μg/mL) | | index | |
| | Toxicity | HSV1 | HSV2 | HSV1 | HSV2 |
| | CC₅₀ | (F) | (MS) | (F) | (MS) |
| GCL | >5 | 0.18 | 0.036 | >28 | >140 |
| Concanavalin A | 29.29 | 5.82 | 5.15 | 5 | 6 |
| Acyclovir | >100 | 0.52^{£} | 2.87^{£} | >192 | >35 |
| Dextran sulfate 8000 | >100 | 15.54 | 4.52 | >6 | >22 |
| Penosan polysulfate | >100 | 4.77 | 2.10 | >21 | >48 |

^{£}concentration, μM

The present invention provides various possibilities of using the lectin in the future by isolating and purifying lectin from Grateloupia chiangii that is a type of red alga not well known to the art, and identifying the utility of the lectin.

Also, the present invention provides a basis for mass-production of the lectin through recombination in the future by identifying an amino acid sequence of the lectin and providing a DNA base sequence coding for the lectin.

While the present invention has been shown and described with reference to certain exemplary embodiments thereof, it will be understood by those skilled in the art that various changes in form and details may be made therein without departing from the spirit and scope of the present invention as defined by the appended claims.

What is claimed is:

1. A method of preparing an antiviral Grateloupia chiangii-derived lectin, comprising:
preparing a crude extract of Grateloupia chiangii that is a red alga; and
purifying a Grateloupia chiangii-derived lectin (GLC) from the crude extract by means of D-mannose affinity chromatography.

2. An antiviral Grateloupia chiangii-derived lectin prepared by the method of claim 1, and having antiviral properties against HSV1 and HSV2.

3. An antiviral Grateloupia chiangii-derived lectin purified and isolated from Grateloupia chiangii, and comprising an amino acid sequence set forth in SEQ ID NO: 1.

4. A lectin gene coding for the antiviral Grateloupia chiangii-derived lectin defined in claim 3, and comprising a base sequence set forth in SEQ ID NO: 2.


SEQUENCE LISTING (2 sequences)

SEQ ID NO: 1 - protein, 231 aa, Artificial Sequence
  GLC
MVVSNRLVSGESLHRGQHIISSDQHFMLVHQYDGNVVLYDVAGSGSGKPLWSTNTYGSHTTVFKMQEDSNLVLYATGGKPIWASDTHGSGGRFLEMQNDGNLVVYTDVGKPKWASDTHQGIAPRSEISVNQVLRIREGLHSANKRNELILQNDGNLVLSKNGSTAWSSGTAGTGANVLVLQIDGNLVLYNDTKSVWASGSMNKGGRTAVMQDDGNFVLYAEGGKPVWATNT

SEQ ID NO: 2 - DNA, 900 bp, Artificial Sequence
  GCL_cDNA
ctctatataacaagcgccagtaatccggttgggatcacagcccagtcatctcagacatcaaccacactagttttccattgaaaattgaaatctctctaacttgtggtgttaacgcaaagaaacatggttgtctccaacagacttgtcagcggtgaaagtttgcatcgtggacaacatataatctcgtctgatcagcattttatgttggtgcatcaatatgatgggaatgtggtcctttacgatgttgccgggagtggctcagggaaacctttatggagcacgaacacatatggttctcacaccactgtctttaaaatgcaagaggattcaaatctcgtgctttatgccactggtgggaaaccgatatgggcatctgacacccacggttccggtggcaggttcctggagatgcaaaatgatggaaatttggttgtgtacaccgacgtaggtaaaccgaaatgggcctcggacacccatcaagggattgcacctcgcagtgaaatctcggtgaatcaagtccttcgcatacgcgaagggctgcattctgcgaataagaggaacgagttgatcttgcagaatgacggaaatttggttctttctaagaatggaagcacagcctggagttctggaactgctggtacgggggctaatgttctggtcctacagattgatggcaacttggtgctttacaacgataccaagagcgtctgggcttctggttcgatgaataagggcggtcgcaccgcggtgatgcaggacgacggcaactttgtgctttacgcggagggaggaaaacccgtctgggctaccaatacgtaataccgcttcgatgccgttccagatatgttcattcactaatgaaagtgatgggataatcgatttaccacgcatatgtagaag